(19) United States
(12) Patent Application Publication
(10) Pub. No.: US 2023/0414884 A1
(43) Pub. Date: Dec. 28, 2023

(54) MINI-INVASIVE SURGICAL NOZZLE, ESPECIALLY FOR INVASIVE SURGERY PURPOSES

(71) Applicant: SKALA-MEDICA S.R.O., Praha (CZ)

(72) Inventors: Radim SKÁLA, Vyhnanice (CZ); Gabriela SKÁLOVÁ, Vyhnanice (CZ); David Hoskovec, Cakovicky (CZ)

(73) Assignee: SKALA-MEDICA S.R.O., Praha (CZ)

(21) Appl. No.: 18/035,617

(22) PCT Filed: Sep. 15, 2021
(86) PCT No.: PCT/CZ2021/000043

§ 371 Date: May 5, 2023

(30) Foreign Application Priority Data
Sep. 24, 2020 (CZ) PUV 2020-38017

(51) Int. Cl. A61M 13/00; A61M 11/02

(52) U.S. Cl. CPC A61M 13/00; A61M 11/02; A61M 2202/064; A61M 2205/7545; A61M 2207/10; A61M 2205/586

(57) ABSTRACT

A mini-invasive surgical nozzle, especially for mini-invasive surgery purposes. The mini-invasive surgical includes a body (7) with first and second ends and a nozzle (6) firmly attached to the first end of the body (7), wherein an outer diameter of the body (7) and an outer diameter of the nozzle (6) attached to the first end are identical. Also, a system including one or more of the mini-invasive surgical nozzles and means for pumping medication through the one or more nozzles into a body cavity of a patient.

### TECHNICAL FIELD

The technical solution refers to a mini-invasive surgical nozzle, especially for mini-invasive surgery purposes, especially for a laparoscopic administration of medicinal substances in the form of aerosol, which facilitates the use in abdominal, thoracic and other cavities, and also allows the administration of gaseous and powdered drugs or mutual substances and aerosols.

### BACKGROUND ART

Currently, products that are aimed at a similar purpose are known, such product enabling the application of drug by spraying, where the medicament is applied by an invasive, laparoscopic procedure. The application pressure is formed using an injector, i.e. a pressure source, such as a peristaltic pump, or an angio-injector of contrast agents.

The product operates on the principle of spraying the medium in the form of a very fine nebula depending on the application pressure and the specific weight of the media. The product comprises critical plastic parts, in particular a tube extended through the nozzle body and locking Bowden cable. These parts do not allow to expose the composition to high temperatures, so that they do not allow the application of hot media as well as low temperatures, which does not allow for example to work with liquid nitrogen. Plastic parts also do not allow a hot air sterilization of the product described with a long-term exposure to temperatures above 130° C.

Known products contain a high pressure nozzle with a movable pin, spring and nozzle orifice with a diameter of 0.15 to 0.2 mm. This solution, due to the use of a high resistance spring and a small diameter of the nozzle orifice, does not allow the formation of aerosol below 0.689 MPa. It also does not allow the application of powder forms of drugs without clogging the nozzle or mutually moving parts of the mechanism.

Due to the inability to operate at low pressure, the existing prior art products do not allow compatibility with previously known, medical peristaltic pumps and medical pumps.

The presence of moving components, in particular a pin having a hub and spring, generally leads to the susceptibility to disorders during the desired function, especially compared to the technical solution based on components firmly associated with the product body. The presence of moving parts leads to lower product life due to wear.

Existing products do not contain a corrosion resistant particle filter with the capabilities of particle capture of over 0.1 mm. They contain an integrated plastic tub extending to the nozzle body. The presence of this tube does not allow efficient cleaning and decontamination of the tool used.

Due to all the above mentioned facts, the existing state of the prior art are exclusively intended for the disposable use without a possibility of re-sterilization.

The existing nozzles exhibit a deteriorated diffusion capacity, usually allow an aerosol scattering only at the angle of 40°. They do not allow the application of liquid medical solutions above 2 ml/second.

The existing solutions do not allow effective operation when connecting 2 to 3 nozzles at the same time by uniform placing them into different positions in the cavity, providing the use of only one source of pressure.

Existing products include an interfering cap nut with a partition wall and space between the nut and the nozzle body, so that the entire product has not a continuous smooth shape that is assumed for the use in laparoscopic surgery. The presence of the cap nut and the partition wall results in impaired hygiene aspects that can cause local sepsis and the deterioration of the patient condition in the event of a capture on the abdominal wall or other organs.

The existing prior art products do not contain an element designed to increase safety and grip ergonomics for the operator.

Existing products have a diameter of about 11 mm in the widest part of the cap nut and contain a distinctive interspace with accompanying edges between the nozzle and the cap nut, which has adverse hygiene aspects.

### SUMMARY OF THE INVENTION

The technical solution of the present invention eliminates the disadvantages of existing products, and its essence consists in that the nozzle is securely attached to one end of the mini-invasive surgical nozzle, wherein the outer diameter of the nozzle body and the outer diameter of the nozzle itself are identical.

A lock for connecting a pressure line with a drug can be welded to the second end of the body of the mini-invasive surgical nozzle.

The body of the mini-invasive surgical nozzle is provided with a ledge having a length of 3 to 10 millimetres before attaching the nozzle. In the nozzle, a filter can be mounted and the nozzle may be provided with a vortex chamber. The nozzle can be provided with a deflector on its mouth. The nozzle exit diameter is in the range of 0.05 mm to 1.5 mm.

The outer surface of the mini-invasive surgical nozzle may be provided with a laser print and a part of the body of the mini-invasive nozzle may be scalloped.

The mini-invasive surgical nozzle according to the technical solution is a medical device intended to spray medical solutions or gases and allows the spraying of temperature and density unstable vapours of hydrogen oxides and liquid nitrogen. Some types allow the spraying of solids drug substances and mixtures thereof in the form of powders having the particle size up to 0.1 mm.

The mini-invasive surgical nozzle allows the medium to be applied in a pressure range of 0.07 MPa-10 MPa. It is a metallic product, a homogeneous composite made of stainless material, which is smooth and continuous along the entire length, and has a circular shape without interfering elements with a maximum of 8 mm diameter.

The mini-invasive surgical nozzle does not contain any plastic parts or moving parts and components, so that the nozzle operation does not depend on the action of a moving pin or spring. The nozzle body design does not contain threaded connections and allows the flow of over 0.000002 m³/s at 2.068 MPa.

The mini-invasive surgical nozzle comprises a corrosion resistant solid particle filter with the capabilities of the mechanical particle capture of from 0.04 mm to 0.2 mm. The product allows operating at negative temperatures and, conversely, at temperatures above the boiling point. It allows the application of gaseous and powdered drugs or mutual substances and aerosols and a steam sterilization, hot air sterilization, plasma sterilization, formaldehyde sterilization, UV-C radiation sterilization, followed by a possible re-sterilization and chemical and mechanical decontamination.

The mini-invasive surgical nozzle is compatible with medical peristaltic pumps and linear dispensers and allows the application of several mini-invasive surgical nozzles at the same time when engaging in one assembly and thus the maximum sector coverage in the treated cavity, while the scalloping ensures the safe and ergonomic grip of this product.

### BRIEF DESCRIPTION OF THE DRAWINGS

An exemplary embodiment of the technical solution is shown in the accompanying drawings, wherein

FIG. 1 shows a schematic system involving two mini-invasive surgical nozzles with a peristaltic pump,

FIG. 2 shows a schematic cross-sectional view at the mini-invasive surgical nozzle with a ledge before welding a nozzle and optionally a lock,

FIG. 3 shows a view at the mini-invasive surgical nozzle prior to welding,

FIG. 4 shows a schematic cross-sectional view at the mini-invasive surgical nozzle with a ledge after welding the nozzle and optionally the lock,

FIG. 5 shows a view at the mini-invasive surgical nozzle after welding,

FIG. 6 shows a view at the mini-invasive surgical nozzle after turning and polishing, including the portion that is provided with scalloping, and

FIG. 7 illustrates schematically a cross-sectional view at the nozzle itself.

### DETAILED DESCRIPTION OF THE INVENTION

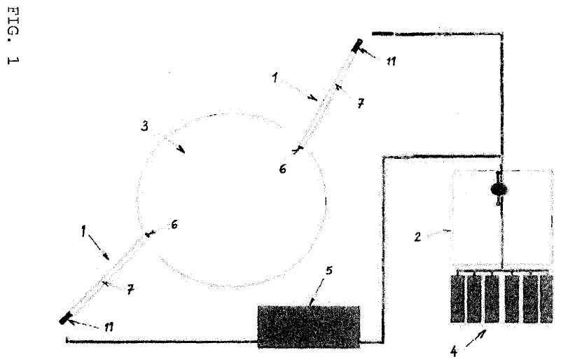

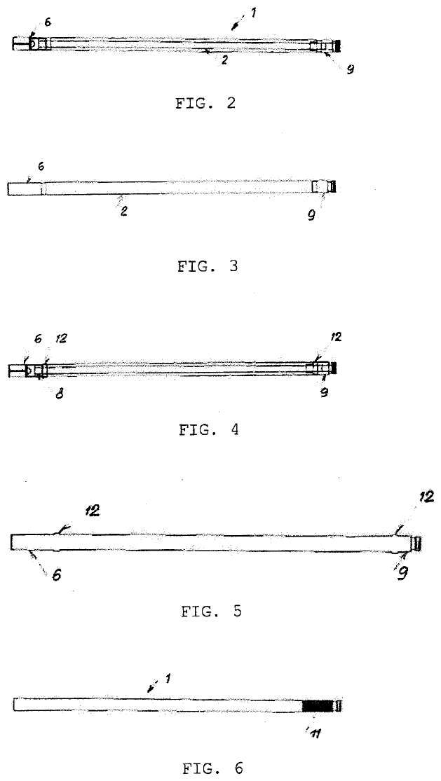

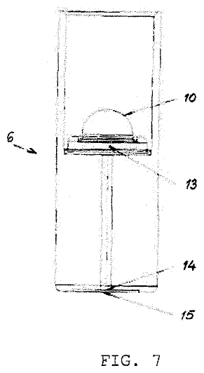

The mini-invasive surgical nozzle 1 is a physician's working tool, which is designed to spray medical solutions and gases, especially in abdominal, thoracic or other cavity 3 in the form of a fine nebula. It is a final part of the system, which is schematically illustrated in FIG. 1, where there is a system involving the implementation of two mini-invasive surgical nozzles 1 with a peristaltic pump 2, wherein various medicinal products are dosed from six packages 4. Subsequently, the drug passes through a pressure line, optionally through a balancing chamber 5, and at the end of which it flows into the mini-invasive surgical nozzle 1, wherein it is sprayed through the nozzle 6 in the form of a fine nebula at the corresponding places in the abdominal, thoracic or gynecologic cavity 3. It is obvious that this system can work with a single mini-invasive surgical nozzle 1, but it is also possible for several mini-invasive surgical nozzles 1 to be involved.

The introduction of mini-invasive surgical nozzles into abdominal, thoracic or other cavities requires an accurate work of the operating physician and allowing variable positions of the mini-invasive surgical nozzles while demanding the most considerate treatment of the patient. All inequalities on the body of the mini-invasive surgical nozzle, including the cup nuts for attaching the nozzle itself cause difficulties and do not allow comfortable work to the physician.

The mini-invasive surgical nozzle 1 according to the technical solution is therefore designed to allow a high variability of the nozzles used 6 and that body 7 of the mini-invasive surgical nozzle 1 should be completely smooth. The mini-invasive surgical nozzle 1 is a cylindrical, metal, homogeneous composite made of stainless, hygienically safe material with diameter generally 8 mm and a length generally over 200 mm.

At one end of the mini-invasive surgical nozzle 1, the nozzle 6 must be connected and at the other end it must be provided with a system connection means that transports active substances into the non-invasive surgical nozzle 1. It is not necessary that both ends of mini-invasive surgical nozzles 1 were designed for connection to the nozzle 6 and the connection to the system in the same way, but such a solution may be advantageous.

At the end of the body 7 of the mini-invasive surgical nozzle 1, where the nozzle 6 is to be connected, a ledge 8 is provided to which the end of the nozzle 6 is co-axial attached and their mutual connection is carried out by welding. The weld 11 must be sized sufficiently so that at the point of connection, on the outer surface of the body 7 of the mini-invasive surgical nozzle 1, no recess occurred, but, by contrast, the weld itself should overlap the outer diameter of the body 7 of the non-invasive surgical nozzle 1. Subsequently, this overlap of the weld 11 is removed by turning and possible grinding or polishing so that the outer surface of the body 7 of the mini-invasive surgical nozzle 1 is completely smooth without any marks after the connection of the nozzle 6 to the body 7 of the mini-invasive surgical nozzle 1.

The respective nozzle 6 has a diameter of the orifice 14 generally in the range of 0.08 mm to 1.5 mm and can be provided with a fixed particle filter 10 with the capabilities of the mechanical particle capture of from 0.04 mm to 0.2 mm. In a particular embodiment, the filter 10 is designed as a cap filter and is connected to the system of vortex chambers 13, which is formed by spiral grooves that distribute the medium evenly. Thus, the medium rotates and thanks to the small cross section on the orifice 14 of the nozzle 6 and the deflector 15, the medium is atomized to very fine drops. As a rule, the spray angle is from 60 to 80° and the spray has a hollow cone shape that can be affected by the shape of the deflector 15 and orifice 14 of the nozzle 6. The shape of the deflector 15 may be different depending on the desired resulting shape of the delicate nebula of the active substance.

The fineness of the aerosol depends on the system pressure. The higher is the pressure the finer drops and greater flow can be achieved. At the same time, a constant pressures across the system is the essential condition.

At the other end of the body 7 of the mini-invasive surgical nozzle 1, a lock 9 may be attached in the same way to connect the mini-invasive surgical nozzle 1 to the system. However, this joint can also be carried out in another known manner for joining these sorts of items.

The body 7 of the mini-invasive surgical nozzle 1 can be preferably provided with a laser printing and some parts can also be provided with scalloping 11 to guarantee a safe, accurate and easy ergonomic grip of the mini-invasive surgical nozzle 1.

The mini-invasive surgical nozzle 1 according to the technical solution allows an easy work of the physician and also provides spraying of temperature and density unstable hydrogen peroxides and liquid nitrogen. It also allows spraying of solids drug substances and mixtures thereof in the form of powders in the particle size up to 0.1 mm.

### INDUSTRIAL APPLICABILITY

The mini-invasive surgical nozzle according to the technical solution is especially useful in the field of a mini-invasive surgery, especially for laparoscopic administration of medicinal substances in the form of aerosol and gases, which facilitates the use in abdominal, thoracic and other cavities and allows the application of gaseous and powdered drugs, or mutual substances and aerosols. In particular, it finds its application in the treatment of malignant cancer diseases in abdominal, thoracic and other cavities where emphasis is placed on the least invasive interventions in the patient, including veterinary care.

### LIST OF REFERENCE NUMERALS

1—mini-invasive surgical nozzle
2—pumping device
3—cavity
4—cover
5—balancing chamber
6—nozzle
7—body
8—ledge
9—lock
10—filter
11—scalloping
12—weld
13—vortex chamber
14—orifice
15—deflector

1. A mini-invasive surgical nozzle, especially for mini-invasive surgery purposes, consisting of a body (7) of the nozzle with two ends and the respective nozzle (6), characterized in that the nozzle (6) is at one end of the body (7) of the mini-invasive surgical nozzle firmly attached, wherein the outer diameter of the body (7) of the respective nozzle and the outer diameter of the respective nozzle (6) are identical.

2. The mini-invasive surgical nozzle, especially for mini-invasive surgery purposes, according to claim 1, characterized in that a lock (9) is welded to the second end of the body (7) of the mini-invasive surgical nozzle for connection of a pressure line with a pharmaceutical.

3. The mini-invasive surgical nozzle, especially for mini-invasive surgery purposes, according to claim 1, characterized in that before attaching the nozzle (6), the body (7) of the mini-invasive surgical nozzle is provided with a ledge (8) having the length of 3 to 10 millimetres.

4. The mini-invasive surgical nozzle, especially for mini-invasive surgery purposes, according to claim 1, characterized in that a filter (10) is mounted in the nozzle (6).

5. The mini-invasive surgical nozzle, especially for mini-invasive surgery purposes, according to claim 1, characterized in that the nozzle (6) is provided with a vortex chamber (13).

6. The mini-invasive surgical nozzle, especially for mini-invasive surgery purposes, according to claim 1, characterized in that the nozzle (6) is provided with a deflector (15) on its mouth (14).

7. The mini-invasive surgical nozzle, especially for mini-invasive surgery purposes, according to claim 1, characterized in that the outlet diameter of the nozzle (6) is in the range of 0.05 mm to 1.5 mm.

8. The mini-invasive surgical nozzle, especially for mini-invasive surgery purposes, according to claim 1, characterized in that the outer surface of the body (7) of the mini-invasive surgical nozzle is provided with a laser print.

9. The mini-invasive surgical nozzle, especially for mini-invasive surgery purposes, according to claim 1, characterized in that a part of the body (7) of the mini-invasive surgical nozzle is provided with scalloping (11).
